(19) United States
(12) Patent Application Publication
(10) Pub. No.: US 2008/0175881 A1
(43) Pub. Date: Jul. 24, 2008

(54) Blood-contacting medical devices for the release of nitric oxide and anti-restenotic agents

(75) Inventors: J. Thomas Ippoliti, St. Paul, MN (US); Scott Schewe, Eden Prairie, MN (US); Michele Zoromski, Minneapolis, MN (US); Robert W. Warner, Woodbury, MN (US); Liliana Atanasoska, Edina, MN (US); Jan Weber, Maastricht (NL)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc.

(21) Appl. No.: 11/654,884

(22) Filed: Jan. 18, 2007

(51) Int. Cl. A61L 31/16; A61L 31/10; A61F 2/82

(52) U.S. Cl. 424/423

(57) ABSTRACT

According to an aspect of the present invention, implantable or insertable blood-contacting devices are provided, which contain one or more release regions that release nitric oxide and one or more anti-restenotic agents. The release region contains one or more polymeric components. The release region also optionally contains one or more inorganic components. Nitric oxide producing groups may be attached to the polymeric component(s), to the optional inorganic component(s), or both. The one or more anti-restenotic agents may be admixed with the polymeric and optional inorganic components, attached to the polymeric component(s), attached to the optional inorganic component(s), or a combination thereof.

### FIELD OF THE INVENTION

The present invention relates generally to medical devices, and more particularly to implantable or insertable medical devices.

### BACKGROUND OF THE INVENTION

Numerous polymer-based medical devices have been developed for implantation or insertion into the body. For example, in recent years, drug eluting coronary stents, which are commercially available from Boston Scientific Corp. (TAXUS), Johnson & Johnson (CYPHER) and others have become the standard of care for maintaining blood vessel patency. These existing products are based on metallic balloon expandable stents with biostable polymer coatings, which release antiproliferative drugs at a controlled rate and total dose, for preventing restenosis of the blood vessel.

CYPHER stents are coated with a thin layer of a blend of poly(n-butyl methacrylate) and ethylene-vinyl acetate copolymer and contain sirolimus as an anti-restenotic agent. R. Virmani et al., Circulation 2004 Feb. 17, 109(6) 701-5. The polymer carrier technology in the TAXUS drug-eluting stent consists of a thermoplastic elastomer poly(styrene-b-isobutylene-b-styrene) (SIBS) with microphase-separated morphology resulting in optimal properties for a drug-delivery stent coating. Physical characterizations of the stent coatings and cast film formulations have shown that paclitaxel exists primarily as discrete nanoparticles embedded in the SIBS matrix. S. Ranade et al., “Physical characterization of controlled release of paclitaxel from the TAXUS™ Express2™ drug-eluting stent,” Journal of Biomedical Materials Research Part A 71A (2004) 625-634.

### SUMMARY OF THE INVENTION

According to an aspect of the present invention, implantable or insertable blood-contacting devices are provided, which comprise a release region that releases both nitric oxide and an anti-restenotic agent. The release region comprises a polymeric component and an optional inorganic component. Nitric oxide producing groups may be attached to the polymeric component, to the optional inorganic component, or both. The release region also comprises an anti-restenotic agent, which may be admixed with the polymeric and optional inorganic component, attached to the polymeric component, attached to the optional inorganic component, or a combination thereof.

An advantage of the present invention is that medical devices can be provided which are capable of releasing an anti-restenotic agent and NO to patients. NO in combination with an anti-restenotic agent (i.e., paclitaxel) has been shown to have a synergistic effect against restenosis. See, e.g., C.-E. Lin et al., “Combination of Paclitaxel and Nitric Oxide as a Novel Treatment for the Reduction of Restenosis,” J. Med. Chem. 2004, 47, 2276-2282.

These and other aspects, embodiments and advantages of the present invention will become immediately apparent to those of ordinary skill in the art upon review of the Detailed Description and Claims to follow.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

According to an aspect of the present invention, implantable or insertable blood-contacting devices are provided, which comprise a release region that releases both nitric oxide and an anti-restenotic agent. The release region comprises a polymeric component and an optional inorganic component. Nitric oxide producing groups may be attached to the polymeric component, to the optional inorganic component, or both. The release region also comprises an anti-restenotic agent, which may be admixed with the polymeric and optional inorganic components, attached to the polymeric component, attached to the optional inorganic component, or a combination thereof.

Examples of blood-contacting medical devices for the practice of the present invention include, for example, stents (e.g., coronary vascular stents and peripheral vascular stents such as cerebral stents and superficial femoral artery (SFA) stents, among others), vascular catheters (including balloon catheters and various central venous catheters), guide wires, balloons, filters (e.g., vena cava filters and mesh filters for distil protection devices), stent coverings, stent grafts, vascular grafts, abdominal aortic aneurysm (AAA) devices (e.g., AAA stents, AAA grafts, etc.), vascular access ports, dialysis ports, embolization devices including cerebral aneurysm filler coils (including Guglilmi detachable coils and metal coils), embolic agents, septal defect closure devices, myocardial plugs, patches, drug depot devices configured for placement in arteries (e.g., for treatment of portions of the artery that lie distal to the device), pacemakers, lead coatings including coatings for pacemaker leads, defibrillation leads and coils, ventricular assist devices including left ventricular assist hearts and pumps, total artificial hearts, shunts, valves including heart valves and vascular valves, anastomosis clips and rings, sutures, suture anchors, tissue staples and ligating clips at surgical sites, cannulae, urethral slings, hernia “meshes”, artificial ligaments, joint prostheses, and tissue engineering scaffolds, among others.

In some embodiments, the release regions of the present invention correspond to an entire medical device. In other embodiments, the release regions correspond to one or more portions of a medical device. For instance, the release regions can be in the form of one or more medical device components, in the form of one or more fibers which are incorporated into a medical device, in the form of one or more layers formed over all or only a portion of an underlying substrate, in the form of one or more plugs that are inserted into a device, and so forth. Materials for use as underlying medical device substrates (where present) include inorganic (e.g., metallic, ceramic, carbon-based, silicon-based, etc.) and organic (e.g., polymeric) substrates. Layers can be provided over an underlying substrate at a variety of locations and in a variety of shapes (e.g., in the form of a series of rectangles, stripes, or any other continuous or non-continuous pattern). As used herein a “layer” of a given material is a region of that material whose thickness is small compared to both its length and width. As used herein a layer need not be planar, for example, taking on the contours of an underlying substrate. Layers can be discontinuous (e.g., patterned).

As used herein, a “release region” is a region (e.g., an entire device, a device component, a device coating layer, a plug, etc.) that comprises a polymeric component and which releases NO and an anti-restenotic agent in vivo. Release regions may comprise, for example, from 25 wt % or less to 50 wt % to 75 wt % to 90 wt % to 95 wt % or more polymeric component. Release regions in accordance with the invention may optionally comprise other components, for example, one or more inorganic components. The release regions may comprise, for example, from 0 wt % to 5 wt % to 10 wt % to 25 wt % to 50 wt % to 75 wt % or more inorganic component.

The polymeric component generally corresponds to a grouping of constitutional units (e.g., 5 to 10 to 25 to 50 to 100 to 250 to 500 to 1000 or more units), commonly referred to as monomers. As used herein, the term “monomers” may refer to the free monomers and those that are incorporated into polymers, with the distinction being clear from the context in which the term is used. The polymeric component may be in the form of a stand-alone polymer, it may be coupled to another entity (e.g., an NO releasing group, an anti-restenotic agent, an optional inorganic component, etc.), and so forth.

The polymeric component may take on a number of configurations, which may be selected, for example, from cyclic, linear and branched configurations, among others. Branched configurations include star-shaped configurations (e.g., configurations in which three or more chains emanate from a single branch point), comb configurations (e.g., configurations having a main chain and a plurality of side chains, also referred to as “graft” configurations), dendritic configurations (e.g., arborescent and hyperbranched polymers), and so forth.

The polymeric component may be a homopolymeric component or a copolymeric component. As used herein, a “homopolymeric component” is a polymeric component that contains multiple copies of a single constitutional unit. As used herein, a “copolymeric component” is a polymeric component that contains multiple copies of at least two dissimilar constitutional units, which may be present, for example, in random, statistical, gradient, periodic (e.g., alternating) or block copolymeric distributions.

Polymeric components may be selected, for example, from suitable members of the following biostable and bioerodable polymers: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflexg); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins and polysaccharides, including fibrin, fibrinogen, collagen, elastin, chitosan, gelatin, starch, and glycosaminoglycans such as hyaluronic acid; as well as further copolymers of the above.

As used herein, a “bioerodable” region is one that loses mass over time as a result of biodegradation and/or other in vivo disintegration processes such as dissolution. As used herein, a “biostable” region, on the other hand, is one characterized by retention of mass over time.

In some embodiments of the invention, the polymeric component contains one or more low glass transition temperature (Tg) polymer blocks and one or more high Tg polymer blocks. As used herein, a “block” or “polymer block” is a grouping of constitutional units (e.g., 5 to 10 to 25 to 50 to 100 to 250 to 500 to 1000 or more units). Blocks can be unbranched or branched. Blocks can contain a single type of constitutional unit (also referred to herein as “homopolymeric blocks”) or multiple types of constitutional units (also referred to herein as “copolymeric blocks”) which may be present, for example, in a random, statistical, gradient, or periodic (e.g., alternating) distribution. As used herein a “chain” is a linear block.

As used herein, a “low Tg polymer block” is one that displays a Tg that is below body temperature, more typically from 35° C. to 20° C. to 0° C. to −25° C. to −50° C. or below. Conversely, as used herein, an elevated or “high Tg polymer block” is one that displays a Tg that is above body temperature, more typically from 40° C. to 50° C. to 75° C. to 100° C. or above. Tg can be measured by differential scanning calorimetry (DSC).

Specific examples of low Tg polymer blocks include homopolymer and copolymer blocks containing one or more of the following (listed along with published Tg's for homopolymers of the same): (1) unsubstituted and substituted alkene monomers including ethylene, propylene (Tg −8 to −13° C.), isobutylene (Tg −73° C.), I-butene (Tg −24° C.), 4-methyl pentene (Tg 29° C.), 1-octene (Tg −63° C.) and other α-olefins, dienes such as 1,3-butadiene, 2-methyl-1,3-butadiene (isoprene), 2,3-dimethyl-1,3-butadiene, 2-ethyl-1,3-butadiene, 1,3-pentadiene, 2-methyl-1,3-pentadiene, 4-butyl-1,3-pentadiene, 2,3-dibutyl-1,3-pentadiene, 2-ethyl-1,3-pentadiene, 1,3-hexadiene, 1,3-octadiene, and 3-butyl-1,3-octadiene, and halogenated alkene monomers including vinylidene chloride (Tg −18° C.), vinylidene fluoride (Tg −40° C.), cis-chlorobutadiene (Tg −20° C.), and trans-chlorobutadiene (Tg −40° C.); (2) acrylic monomers including: (a) alkyl acrylates such as methyl acrylate (Tg 10° C.), ethyl acrylate (Tg −24° C.), propyl acrylate, isopropyl acrylate (Tg −11° C., isotactic), butyl acrylate (Tg −54° C.), sec-butyl acrylate (Tg −26° C.), isobutyl acrylate (Tg −24° C.), cyclohexyl acrylate (Tg 19° C.), 2-ethylhexyl acrylate (Tg −50° C.), dodecyl acrylate (Tg −3° C.) and hexadecyl acrylate (Tg 35° C.), (b) arylalkyl acrylates such as benzyl acrylate (Tg 6° C.), (c) alkoxyalkyl acrylates such as 2-ethoxyethyl acrylate (Tg −50° C.) and 2-methoxyethyl acrylate (Tg −50° C.), (d) halo-alkyl acrylates such as 2,2,2-trifluoroethyl acrylate (Tg −10° C.) and (e) cyano-alkyl acrylates such as 2-cyanoethyl acrylate (Tg 4° C.); (3) methacrylic monomers including (a) alkyl methacrylates such as butyl methacrylate (Tg 20° C.), hexyl methacrylate (Tg −5° C.), 2-ethylhexyl methacrylate (Tg −10° C.), octyl methacrylate (Tg −20° C.), dodecyl methacrylate (Tg −65° C.), hexadecyl methacrylate (Tg 15° C.) and octadecyl methacrylate (Tg −100° C.) and (b) aminoalkyl methacrylates such as diethylaminoethyl methacrylate (Tg 20° C.) and 2-tert-butyl-aminoethyl methacrylate (Tg 33° C.); (4) vinyl ether monomers including (a) alkyl vinyl ethers such as methyl vinyl ether (Tg −31° C.), ethyl vinyl ether (Tg −43° C.), propyl vinyl ether (Tg −49° C.), butyl vinyl ether (Tg −55° C.), isobutyl vinyl ether (Tg −19° C.), 2-ethylhexyl vinyl ether (Tg −66° C.) and dodecyl vinyl ether (Tg −62° C.); (5) cyclic ether monomers include tetrahydrofuran (Tg −84° C.), trimethylene oxide (Tg −78° C.), ethylene oxide (Tg −66° C.), propylene oxide (Tg −75° C.), methyl glycidyl ether (Tg −62° C.), butyl glycidyl ether (Tg −79° C.), allyl glycidyl ether (Tg −78° C), epibromohydrin (Tg −14° C.), epichlorohydrin (Tg −22° C.), 1,2-epoxybutane (Tg −70° C.), 1,2-epoxyoctane (Tg −67° C.) and 1,2-epoxydecane (Tg −70° C.); (6) ester monomers (other than the above acrylates and methacrylates) including ethylene malonate (Tg −29° C.), vinyl acetate (Tg 30° C.), and vinyl propionate (Tg 10° C.); and (7) siloxane monomers including dimethylsiloxane (Tg −127° C.), diethylsiloxane, methylethylsiloxane, methylphenylsiloxane (Tg −86° C.), and diphenylsiloxane.

Specific examples of high Tg polymer blocks include homopolymer and copolymer blocks containing one or more of the following: (1) vinyl aromatic monomers including (a) unsubstituted vinyl aromatics, such as styrene (Tg 100° C.) and 2-vinyl naphthalene (Tg 151° C.), (b) vinyl substituted aromatics such as alpha-methyl styrene, and (c) ring-substituted vinyl aromatics including ring-alkylated vinyl aromatics such as 3-methylstyrene (Tg 97° C.), 4-methylstyrene (Tg 97° C.), 2,4-dimethylstyrene (Tg 112° C.), 2,5-dimethylstyrene (Tg 143° C.), 3,5-dimethylstyrene (Tg 104° C.), 2,4,6-trimethylstyrene (Tg 162° C.), and 4-tert-butylstyrene (Tg 127° C.), ring-alkoxylated vinyl aromatics, such as 4-methoxystyrene (Tg 113° C.) and 4-ethoxystyrene (Tg 86° C.), ring-halogenated vinyl aromatics such as 2-chlorostyrene (Tg 119° C.), 3-chlorostyrene (Tg 90° C.), 4-chlorostyrene (Tg 110° C.), 2,6-dichlorostyrene (Tg 167° C.), 4-bromostyrene (Tg 118° C.) and 4-fluorostyrene (Tg 95° C.), ring-ester-substituted vinyl aromatics such as 4-acetoxystyrene (Tg 116° C.), ring-hydroxylated vinyl aromatics such as 4-hydroxystyrene (Tg 174° C.), ring-amino-substituted vinyl aromatics including 4-amino styrene, ring-silyl-substituted styrenes such as p-dimethylethoxy siloxy styrene, unsubstituted and substituted vinyl pyridines such as 2-vinyl pyridine (Tg 104° C.) and 4-vinyl pyridine (Tg 142° C.), and other vinyl aromatic monomers such as vinyl carbazole (Tg 227° C.) and vinyl ferrocene (Tg 189° C.); (2) other vinyl monomers including (a) vinyl esters such as vinyl benzoate (Tg 71° C.), vinyl 4-tert-butyl benzoate (Tg 101° C.), vinyl cyclohexanoate (Tg 76° C.), vinyl pivalate (Tg 86° C.), vinyl trifluoroacetate (Tg 46° C.), vinyl butyral (Tg 49° C.), (b) vinyl amines, (c) vinyl halides such as vinyl chloride (Tg 81° C.) and vinyl fluoride (Tg 40° C.), (d) alkyl vinyl ethers such as tert-butyl vinyl ether (Tg 88° C.) and cyclohexyl vinyl ether (Tg 81° C.), and (e) other vinyl compounds such as vinyl pyrrolidone; (3) other aromatic monomers including acenaphthalene (Tg 214° C.) and indene (Tg 85° C.); (4) methacrylic monomers including (a) methacrylic acid anhydride (Tg 159° C.), (b) methacrylic acid esters (methacrylates) including (i) alkyl methacrylates such as methyl methacrylate (Tg 105-120° C.), ethyl methacrylate (Tg 65° C.), isopropyl methacrylate (Tg 81° C.), isobutyl methacrylate (Tg 53° C.), t-butyl methacrylate (Tg 118° C.) and cyclohexyl methacrylate (Tg 92° C.), (ii) aromatic methacrylates such as phenyl methacrylate (Tg 110° C.) and including aromatic alkyl methacrylates such as benzyl methacrylate (Tg 54° C.), (iii) hydroxyalkyl methacrylates such as 2-hydroxyethyl methacrylate (Tg 57° C.) and 2-hydroxypropyl methacrylate (Tg 76° C.), (iv) additional methacrylates including isobornyl methacrylate (Tg 110° C.) and trimethylsilyl methacrylate (Tg 68° C.), and (c) other methacrylic-acid derivatives including methacrylonitrile (Tg 120° C.); (5) acrylic monomers including (a) certain acrylic acid esters such as tert-butyl acrylate (Tg 43-107° C.), hexyl acrylate (Tg 57° C.) and isobornyl acrylate (Tg 94° C.); and (b) other acrylic-acid derivatives including acrylonitrile (Tg 125° C.).

For example, as used herein, a poly(vinyl aromatic) block is a polymer block that contains multiple copies of one or more types of vinyl aromatic monomers, a polyalkene block is a block that contains multiple copies of one or more types of alkene monomers, a polyacrylate block is a block that contains multiple copies of one or more types of acrylate monomers, a polysiloxane block is a block that contains multiple copies of one or more types of siloxane monomers, and so forth.

Block copolymeric configurations may vary widely and include, for example, the following configurations, among others, which comprise two more high Tg polymer chains (designated “H”) and one or more low Tg polymer chains (designated “L”): (a) block copolymers having alternating chains of the type HLH, (HL)ₘ, (LH)ₘ, L(HL)ₘ and H(LH)ₘ where m is a positive whole number of 2 or more, (b) multiarm (including star) copolymers such as X(LH)ₘ, where X is a hub species (e.g., an initiator molecule residue, a linking residue, etc.), and (c) comb copolymers having an L chain backbone and multiple H side chains.

Polymers of this type are capable of demonstrating high strength and elastomeric properties, while at the same time being processable using techniques such as solvent—and/or melt-based processing techniques. As is well known, block copolymers tend to phase separate. In the polymers like those described above, the high Tg blocks (which are hard) will aggregate to form hard phase domains. Without wishing to be bound by theory, where high Tg hard blocks are interconnected via low Tg blocks (or portions thereof, e.g., in the case of a comb copolymer, which low Tg blocks or portions thereof are elastomeric), the hard phase domains may become physically crosslinked to one another via the elastomeric blocks. Moreover, because the crosslinks are not covalent in nature, they can be reversed, for example, by dissolving or melting the block copolymer.

As will be appreciated by those of ordinary skill in the art, polymers and copolymers employed in accordance with the present invention may be synthesized according to known methods, including cationic, anionic, and radical polymerization methods, particularly controlled/“living” cationic, anionic and radical polymerizations.

In this regard, living cationic polymerization of unsaturated monomers, including alkenes such as isobutylene, butadiene, isoprene, methylbutene, and 2-methylpentene, among others, or vinyl aromatic monomers, such as styrene, p-methylstyrene, alpha-methylstyrene and indene, among others, is well known. In a typical cationic polymerization process, a suitable unsaturated monomer is polymerized in the presence of a cationic polymerization catalyst, an initiator, and an optional Lewis base (in order to prevent initiation by protic impurities), typically in an aprotic solvent under dry conditions at low temperature. The polymers formed in this method are living cationic polymers (e.g., polymers in which the polymer chains typically continue to grow from the site of initiation until the monomer supply is exhausted, rather than terminating when the chain reaches a certain length or when the catalyst is exhausted). The cationic polymerization catalyst may be, for example, a Lewis acid (e.g., BCl₃ or TiCl₄, among others). The initiator may be, for example, an alkyl halide or (haloalkyl)-aryl compound, for example, a monofunctional initiator such as 2-chloro-2,4,4-trimethylpentane, a bifunctional initiator such as 1,3-di(1-chloro-1-methylethyl)-5-(t-butyl)benzene, or a trifunctional initiator such as 1,3,5-tri(1-chloro-1-methylethyl)benzene, among others. Lewis bases include pyridine and its derivatives, such as 2,6-ditert-butyl-pyridine (DTBP) or lutidine, among others.

Living free radical polymerizations may be employed in various embodiments, due to the undemanding nature of radical polymerizations in combination with the power to control polydispersity, architecture, and molecular weight that living processes provide. Monomers capable of free radical polymerization vary widely and may be selected from the following, among many others: vinyl aromatic monomers such as substituted and unsubstituted styrene, diene monomers such as 1,3-butadiene, chloroprene, isoprene and p-divinylbenzene, acrylate monomers, for example, acrylate esters such as butyl acrylate and methyl acrylate, methacrylate monomers, for example, methacrylic esters such as methyl methacrylate, beta-hydroxyethyl methacrylate, beta-dimethylaminoethyl methacrylate and ethylene glycol dimethacrylate, as well as other unsaturated monomers including acrylic acid, acrylamide, acrylonitrile, ethylene, propylene, tetrafluoroethylene, triflourochloroethylene, iraconic acid, fumaric acid, maleic acid, methacrylic acid, methacrylonitrile, vinyl esters such as vinyl acetate, vinyl chloride, vinyl fluoride, N-vinylpyrrolidinone, N-vinylimidazole, vinylidene chloride, vinylidene fluoride and N,N′-methylenebis-acrylamide, among many others.

Specific examples of free radical polymerization processes include metal-catalyzed atom transfer radical polymerization (ATRP), stable free-radical polymerization (SFRP), including nitroxide-mediated processes (NMP), and degenerative transfer including reversible addition-fragmentation chain transfer (RAFT) processes. These methods are well-detailed in the literature and are described, for example, in an article by Pyun and Matyjaszewski, “Synthesis of Nanocomposite Organic/Inorganic Hybrid Materials Using Controlled/“Living” Radical Polymerization,” Chem. Mater., 13:3436-3448 (2001), B. Reeves, “Recent Advances in Living Free Radical Polymerization,” Nov. 20, 2001. University of Florida, T. Kowalewski et al., “Complex nanostructured materials from segmented copolymers prepared by ATRP,” Eur. Phys. J. E, 10, 5-16 (2003).

ATRP is a particularly appealing free radical polymerization technique, as it is tolerant of a variety of functional groups (e.g., alcohol, amine, and sulfonate groups, among others) and thus allows for the polymerization of many monomers. In monomer polymerization via ATRP, radicals are commonly generated using organic halide initiators and transition-metal complexes. Some typical examples of organic halide initiators include alkyl halides, haloesters (e.g., methyl 2-bromopropionate, ethyl 2-bromoisobutyrate, etc.) and benzyl halides (e.g., 1-phenylethyl bromide, benzyl bromide, etc.), among others. A wide range of transition-metal complexes may be employed, including a variety of Cu-, Ru-, Os- and Fe-based systems, among others. Examples of monomers that may be used in ATRP polymerization reactions include various unsaturated monomers such as alkyl methacrylates, alkyl acrylates, hydroxyalkyl methacrylates, vinyl esters, vinyl aromatic monomers, acrylamides, methacrylamides, acrylonitrile, and 4-vinylpyridine, among others. In ATRP, at the end of the polymerization, the polymer chains are capped with a halogen atom that can be readily transformed via S_{N}1, S_{N}2 or radical chemistry to provide other functional groups such as amino groups, among many others. Functionality can also be introduced into the polymer by other methods, for example, by employing initiators that contain functional groups which do not participate in the radical polymerization process. Examples include initiators with epoxide, azido, amino, hydroxyl, cyano, and allyl groups, among others. In addition, functional groups may be present on the monomers themselves.

Using the above and other polymerization techniques, various strategies may be employed for forming polymers, including various block copolymers, for use in accordance with the invention. Examples include successive monomer addition (a) from a mono- or di-functional intiator (e.g., for linear AB and BAB type block copolymers, respectively) and (b) tri-, quatra-, penta-, etc. functional initiators (e.g., for the formation of star copolymers), among others.

Multiple types of polymerization techniques may also be employed to form a single type of block copolymer. For example, radical polymerization techniques may be employed in connection with polymer blocks that contain monomers which are not radically polymerizable, such as isobutylene, among others. In this regard, macroinitiators may be prepared using non-free-radical techniques, such as living anionic or cationic techniques by appropriate modification of the end groups of the resulting polymers, for instance, by the introducing at least one radically transferable atom, such as those found in halide groups such as benzylic halide and a-halo ester groups, among others. As another example, functional initiators (which may be protected) may be employed for a first type of polymerization process, followed by deprotection/conversion of the functional group(s), as needed, followed by polymerization via a second polymerization process.

Classes of nitric oxide donors for use in the present invention include suitable members of the following, among others: organic nitrates, organic nitrites, metal-NO complexes, N-nitrosamines, N-hydroxy-N-nitrosamines, N-nitrosimines, nitrosothiols, C-nitroso compounds, diazetine dioxides, furoxans including benzofuroxans, oxatriazole-5-imines, sydnonimines, oximes, hydroxylamines, N-hydroxyguanidines and hydroxyurea. These are described in more detail in P. G. Wang et al., “Nitric Oxide Donors: Chemical Activities and Biological Applications,” Chem. Rev., 102 (2002) 1091-1134. Typical loadings range from less than or equal to 5 wt % to 10 wt % to 15 wt % to 20 wt % to 25 wt % to 30 wt % or more.

Nitric oxide producing groups may be covalently attached to the polymeric component of the release region, in some embodiments of the invention. For example, in certain embodiments, zwitterionic N-diazeniumdiolates are prepared by exposing diamine-containing compounds to NO at elevated pressure (e.g., 5 atm). This reaction has been represented as follows:

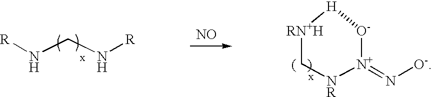

In other embodiments, anionic diazeniumdiolates may be prepared from secondary amines by adding a basic salt, such as sodium methoxide, during the NO addition process. This reaction has been represented as follows:

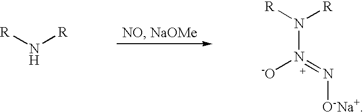

Exposure of such diazeniumdiolates to hydrogen donors (e.g. water under physiological conditions) is known to stimulate NO release. For further information, see, e.g., M. M. Reynolds et al. infra as well as the references cited therein.

Using the above and other nitric oxide donors, nitric oxide producing polymers may be prepared. For example, nitric oxide donors may be provided within poly(isobutylene-co-styrene) polymers. In a first scheme, a copolymer of styrene, isobutylene, and bromomethyl-substituted styrene,

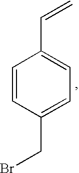

is prepared using standard cationic polymerization techniques, followed by reaction with a diamine

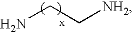

where x is an integer, to form an amine-substituted poly(isobutylene-co-styrene), molecule 2, represented schematically as follows:

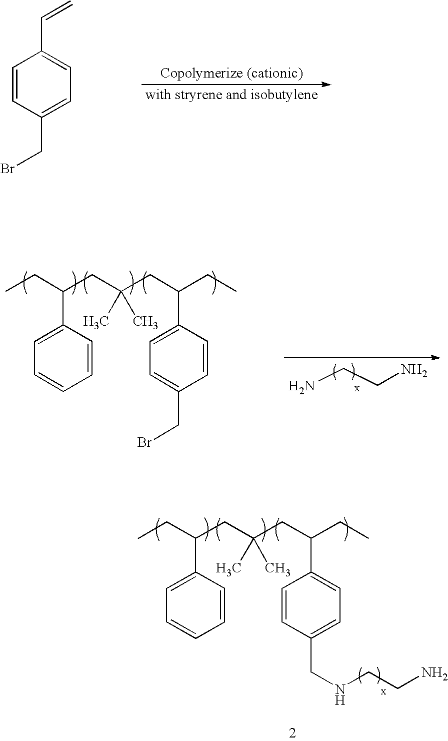

For example, isobutylene polymerization may proceed from a difunctional initiator, followed by polymerization of the styrene monomers (e.g., an admixture of styrene and bromomethyl-substituted styrene may be polymerized, or styrene may first be polymerized followed by polymerization of bromomethyl-substituted styrene, or bromomethyl-substituted styrene may first be polymerized followed by polymerization of styrene), followed by reaction with a diamine, thereby forming an amine substituted poly(styrene-b-isobutylene-b-styrene)-type triblock copolymer (note that this nomenclature disregards the presence of the initiator in the center of the isobutylene block) in which diamine groups are attached to some of the styrene monomers.

In a second scheme, a ring-diamine-substituted styrene monomer

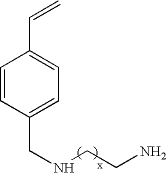

is formed by reacting a bromomethyl-substituted styrene,

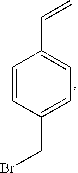

with a diamine,

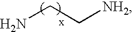

This monomer, however, cannot be polymerized cationically due to the presence of the primary amine. However, it can be polymerized using ATRP to produce an amine-substituted poly(isobutylene-co-styrene) molecule 2 as represented schematically in the following scheme:

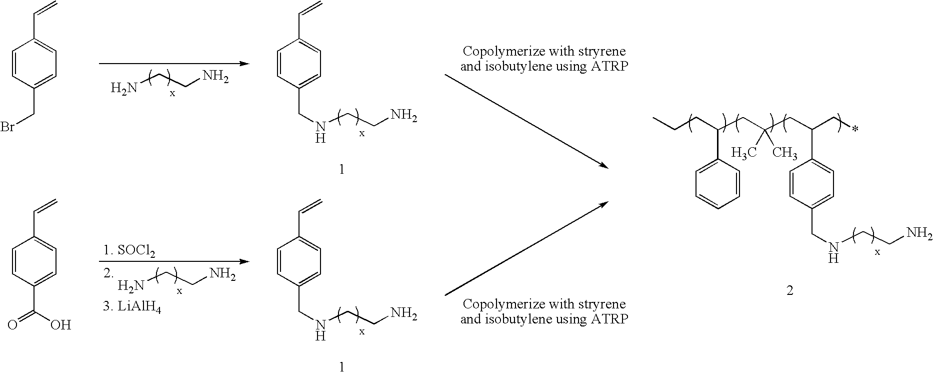

Polymerization may proceed, for example, using a modification of a procedure reported by Jankova et al., “Synthesis of poly(styrene-b-isobutylene-b-styrene) triblock copolymer by ATRP,” Polymer Bulletin 41 (1998) 639-644, in which polyisobutylene (which cannot be polymerized by ATRP) is functionalized with phenol at both ends and reacted with 2-bromopropionyl chloride to form a macroinitiator for ATRP. The synthesized difunctional polyisobutylene macroinitiator is subsequently heated with a solution of styrene in xylene under conditions for ATRP using CuBr/bipyridine as a copper coordination complex, thereby forming polystyrene blocks at each end of the macroinitiator.

In the present invention, an analogous procedure may be used. For instance, an admixture of styrene and amine-substituted styrene may be copolymerized in the presence of a difunctional polyisobutylene macroinitiator in accordance with this scheme, or styrene may first be polymerized in the presence of a difunctional polyisobutylene macroinitiator followed by polymerization of amine-substituted styrene, or amine-substituted styrene may first be polymerized followed by polymerization of styrene.

Regardless of the scheme for forming the amine-substituted poly(isobutylene-co-styrene) polymer, this polymer may now be converted to an N-diazeniumdiolate-modified polymer capable of releasing NO by exposing the polymer to NO at elevated pressure (e.g. 5 atm):

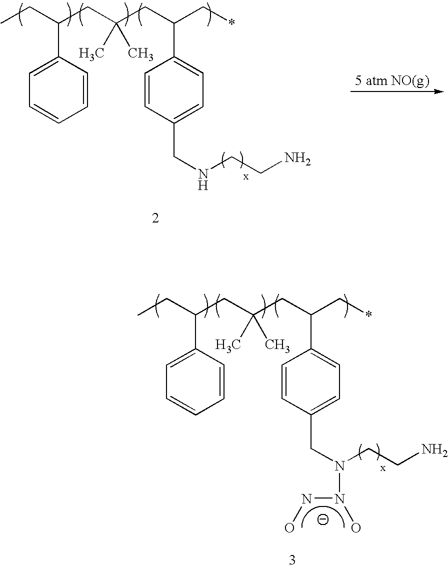

As another example, nitric oxide donors may be provided within polysaccharides via polysaccharide-amine conjugates such as those described in T. Azzam et al. “Polysaccharide-Oligoamine Based Conjugates for Gene Delivery,” J. Med. Chem., 45 (2002) 1817-1824. This reference describe methods for conjugating various amines, including spermine, spermidine, N,N′-bis(3-aminopropyl)-1,3-propanediamine, N,N′-bis(3-aminopropyl)ethylenediamine, N′N′-bis(2-aminoethyl)-1,3-propanediamine, polyethyleneimine, ethanediamine, 1,3-propanediamine, butanediamine, hexanediamine, octanediamine, triethylene glycol diamine, diethylenetriamine, N,N-dimethylpropylenediamine, and N,N-dimethylethylenediamine, to various polysaccharides by reductive amination of oxidized polysaccharides with the desired amine, followed by reduction to stable amine conjugates using sodium borohydride.

According to an embodiment of the invention, polysaccharide-amine conjugates thus obtained may be converted to N-diazeniumdiolates as described above. Such processes may be applied to polysaccharide homopolymers or to block copolymers having at least one polysaccharide block and at least one differing polymer block, for example, selected from those set forth above, among others.

In addition to the above NO-releasing polysaccharides, other NO-releasing bioerodable polymers may be employed. See, e.g., A. P. Bonartsev et al., “A New System of Nitric Oxide Donor Prolonged Delivery on Basis of Controlled-Release Polymer, Polyhydroxybutyrate,” AJH—May 2005—Vol. 18, NO. 5, Part 2, Posters: Antihypertensive Drugs and Pharmacology, p. 51A.

Other examples of NO releasing block polymers for the practice of the present invention include the NO releasing (diazeniumdiolate-modified) polyurethanes described in H.-W. Jun et al., “Nitric Oxide-Producing Polyurethanes,” Biomacromolecules 6 (2005) 838-844.

Various additional polymers with covalently linked NO donors that have been formed include (a) diazeniumdiolated 1,4-butanediol-diglycidyl-ether-crosslinked poly(ethyleneimine), (b) diazeniumdiolated diamino-crosslinked polydimethoxysilane, (c) diazeniumdiolated polymethacrylate-based homo- and co-polymers that contain linear and cyclic pendant secondary amine sites, and (d) methoxymethyl-protected diazeniumdiolated piperazine poly(vinyl chloride). In the latter case, nitric oxide release from the polymer was shown to be very slow due to the slow rate of hydrolysis of the protecting group. For further details, see, e.g., M. M. Reynolds et al., “Nitric Oxide-Releasing Hydrophobic Polymers: Preparation, Characterization, and Potential Biomedical Applications,” Free Radical Biology & Medicine, 37 (2004) 926-936, Frost et al., “Polymers incorporating nitric oxide releasing/generating substances for improved biocompatibility of blood-contacting medical devices,” Biomaterials 26 (2005) 1685-1693, P. G. Parzuchowski et al., “Synthesis and Characterization of Polymethacrylate-Based Nitric Oxide Donors,” J Am. Chem. Soc. 2002, 124, 12182-12191, K. A. Mowery et al., “Preparation and characterization of hydrophobic polymeric films that are thromboresistant via nitric oxide release,” Biomaterials 21 (2000) 9-21, and the references cited therein.

As noted above, in some embodiments, the release regions of the devices of the invention may comprise an optional inorganic component.

In some embodiments, the inorganic component comprises particles. Particles for use in the present invention can vary widely in shape and size. In some instances, the particles are nanoparticles, which are particles that have at least one major dimension (e.g., the thickness for a nanoplates, the diameter for a nanospheres, nanocylinders and nanotubes, etc.) that is less than 1000 nm, more typically less than 100 nm. Hence, for example, nanoplates typically have at least one dimension (e.g., thickness) that is less than 1000 nm, other nanoparticles typically have at least two orthogonal dimensions (e.g., thickness and width for nano-ribbons, diameter for cylindrical and tubular nanoparticles, etc.) that are less than 1000 nm, while still other nanoparticles typically have three orthogonal dimensions that are less than 1000 nm (e.g., the diameter for nanospheres). A wide variety of nanoparticles are known including, for example, carbon, ceramic and metallic nanoparticles including nanoplates, nano-ribbons, nanotubes, and nanospheres, and other nanoparticles. Specific examples of nanoplates include synthetic or natural phyllosilicates including clays and micas (which may optionally be intercalated and/or exfoliated) such as montmorillonite, hectorite, hydrotalcite, vermiculite and laponite. Specific examples of nanotubes and nanofibers include single-wall, so-called “few-wall,” and multi-wall carbon nanotubes, vapor grown carbon fibers, alumina nanofibers, titanium oxide nanofibers, tungsten oxide nanofibers, tantalum oxide nanofibers, zirconium oxide nanofibers, and silicate nanofibers such as aluminum silicate nanofibers. Specific examples of further nanoparticles (e.g., nanoparticles having three orthogonal dimensions that are less than 1000 nm) include fullerenes (e.g., “Buckey balls”), silica nanoparticles, gold nanoparticles, aluminum oxide nanoparticles, titanium oxide nanoparticles, tungsten oxide nanoparticles, tantalum oxide nanoparticles, zirconium oxide nanoparticles, and monomeric silicates such as polyhedral oligomeric silsequioxanes (POSS), including various functionalized POSS and polymerized POSS.

In some embodiments, the inorganic component may comprise a sol-gel-generated ceramic component. By way of background, it is well known that ceramic regions may be formed using sol-gel processing. In a typical sol-gel process, precursor materials, typically selected from inorganic metallic and semi-metallic salts, metallic and semi-metallic complexes/chelates, metallic and semi-metallic hydroxides, and organometallic and organo-semi-metallic compounds such as metal alkoxides and alkoxysilanes, are subjected to hydrolysis and condensation (also referred to sometimes as polymerization) reactions, thereby forming a “sol” (i.e., a suspension of solid particles within a liquid). For example, an alkoxide of choice (such as a methoxide, ethoxide, isopropoxide, tert-butoxide, etc.) of a semi-metal or metal of choice (such as silicon, germanium aluminum, zirconium, titanium, tin, iron, hafnium, tantalum, molybdenum, tungsten, rhenium, iridium, etc.) may be dissolved in a suitable solvent, for example, in one or more alcohols. Subsequently, water or another aqueous solution such as an acidic or basic aqueous solution (which aqueous solution can further contain organic solvent species such as alcohols) is added, causing hydrolysis and condensation to occur. Further processing of the sol enables solid materials to be made in a variety of different forms. For instance, “wet gel” coatings can be produced by spray coating, coating with an applicator (e.g., by roller or brush), ink-jet printing, screen printing, and so forth. The wet gel is then dried to form a ceramic region. Further information concerning sol-gel materials can be found, for example, in Viitala R. et al., “Surface properties of in vitro bioactive and non-bioactive sol-gel derived materials,” Biomaterials, 2002 August; 23(15):3073-86.

Polymer-ceramic composite (hybrid) regions may be formed based upon analogous processes, as well as upon principles of polymer synthesis, manipulation, processing, and so forth. Sol gel processes are suitable for use in conjunction with polymers and their precursors, for example, because they can be performed at ambient temperatures. A review of various techniques for generating polymeric-ceramic composites can be found, for example, in G. Kickelbick, “Concepts for the incorporation of inorganic building blocks into organic polymers on a nanoscale,” Prog. Polym. Sci., 28 (2003) 83-114.

It is known, for example, to impregnate a gel such as a xerogel with monomer and polymerize the monomer within the gel. Best results are usually obtained where interactions between the monomer/polymer and the gel are sufficiently strong to prevent macroscopic phase separation. Conversely, it is also known, for example, to generate polymeric-ceramic composites by conducting sol gel processing in the presence of a preformed polymer, which techniques tend to be successful, for example, where the polymer is soluble in the sol-forming solution and/or where the polymer has substantial interactions with the ceramic phase (e.g., due to hydrogen bonding between hydroxyl groups and electronegative atoms within the polymeric and ceramic phases, etc.), which prevent macroscopic phase separation. One way of improving the interactions between the polymeric and ceramic components is to employ a charged polymer, or ionomer. For this purpose, polymers may be functionalized with anionic groups, such as sulfonate or carboxylate groups, among others, or cationic groups, such as ammonium groups, among others.

Nanoscale phase domains may also be achieved by providing covalent interactions between the polymeric and ceramic phases. This result can be achieved via a number of known techniques, including the following: (a) providing species with both polymer and ceramic precursor groups and thereafter conducting polymerization and hydrolysis/condensation simultaneously, (b) providing polymers with ceramic precursor groups (e.g., groups that are capable of participation in hydrolysis/condensation, such as metal or semi-metal alkoxide groups), followed by hydrolysis/condensation of the precursor groups and (c) providing a ceramic sol with polymer precursor groups (e.g., groups that are capable of participation in a polymerization reaction, such as vinyl groups or cyclic ether groups) and thereafter conducting one or more polymerization steps.

Similarly, it is also known to provide nanoparticles (e.g., carbon nanotubes, etc.) with polymer precursor groups (e.g., groups that are capable of participation in a polymerization reaction, such as vinyl groups or cyclic ether groups) and thereafter conduct one or more polymerization steps.

In certain embodiments of the invention, nitric oxide producing groups are attached to the optional inorganic component.

For example, nitric oxide releasing carbon nanotubes may be formed. In this regard, processes for forming amide and amine functionalized nanotubes are described in T. Ramanathan et al., “Amino-Functionalized Carbon Nanotubes for Binding to Polymers and Biological Systems,” Chem. Mater., 17 (2005) 1290-1295. These methods involve either (a) reduction of carboxyl groups to hydroxymethyl groups, followed by transformation into aminomethyl groups or (b) direct coupling of diamine (e.g., ethylene diamine) with carboxylic groups to introduce amine groups via amide linkages. In the latter case, other amines besides ethylene diamine may be employed including suitable members of those discussed in conjunction with Azzam et al. supra. These amine-functionalized nanotubes may then be loaded with NO as described above, thereby forming N-diazeniumdiolates.

Nitric oxide releasing carbon nanotubes may also be formed by reacting hydroxy-functionalized carbon nanotubes with alkoxyaminosilanes such as those described in Marxer et al. et al. infra (e.g., AEMP3, AHAP3, DET3 or AEAP2, among others). An analogous procedure would be one in which hydroxy-functionalized carbon nanotubes are reacted with an alkoxysilane. Functionalized carbon nanotubes may be incorporated into polymer composites using various techniques, for example, by in situ polymerization in the presence of the nanotubes or by solution mixing of the nanotubes with one or more polymers. See Zhang et al, Sensors and Actuators B109, 2005, 323. Once the amines are attached to the nanotubes, they may be loaded with NO as described above, thereby forming N-diazeniumdiolates.

As another example, nitric oxide releasing metallic particles, specifically gold particles, are known from A. R. Rothrock et al., “Synthesis of Nitric Oxide-Releasing Gold Nanoparticles,” J. Am. Chem. Soc., 127 (2005) 9362-9363.

As yet another example, M. M. Reynolds et al. and Frost et al. supra describe reacting ceramic particles, specifically fumed silica, with different mono- and di-amine alkoxysilane reagents, i.e., aminoalkoxysilanes such as (CH₃O)₃Si(CH₂)₃NHR, where R═—H, —CH₃, —(CH₂)₂NH₂ or —(CH₂)₆NH₂. These may be reacted with NO under elevated pressure in the presence of base to form N-diazeniumdiolate groups. These authors also report tethering nitrosothiols to the surface of fumed silica filler particles. In this process, a primary-amine-containing silane reagent (e.g., (CH₃O)₃Si(CH₂)₃NH₂) is first attached to the silica. The terminal amine is then reacted with a self-protected thiolactone of N-acetylpenicillamine, forming an amide bond and yielding a free sulfhydryl group on the surface of the particles. This group can then be reacted with t-butylnitrite to form S-nitroso-N-acetylpenicillamine (SNAP) derivatized fumed silica particles. Nitric oxide release may be triggered by light, which photolytically cleaves the S—N bond. For example, NO release may be triggered in this fashion from medical devices that lie close to the skin (e.g., SFA stents, etc.) as there is a high risk of restenosis in these areas.

Nitric oxide releasing sol gels are described in Marxer et al., “Preparation of Nitric Oxide (NO)-Releasing Sol-Gels for Biomaterial Applications,” Chem. Mater. 15 (2003) 4193-4199 and in Nablo et al., “Antibacterial properties of nitric oxide-releasing sol-gels,” Journal of Biomedical Materials Research Part A, 67A (2003) 1276-1283. Such gels may be formed by first forming an amine-containing sol-gel using an alkylalkoxysilane such as isobutyltrimethoxysilane (BTMOS) and an alkoxyaminosilane such as aminoethylaminomethylphenethyltrimethoxysilane (AEMP3), N-(6-aminohexyl)-aminopropyltrimethoxysilane (AHAP3), N-(3 -trimethoxysilylpropyl)-diethylenetriamine (DET3) or N-(2-aminoethyl)-3-aminopropylmethyldimethoxysilane (AEAP2). Reaction of NO with the secondary diamines (e.g., at elevated NO pressure) produces diazeniumdiolate NO donors.

If desired, amine-containing sol-gel polymer hybrids may be formed using alkoxyaminosilanes such as those in the prior paragraph in conjunction with techniques analogous to those discussed above (e.g., providing species with both polymer and ceramic precursor groups and thereafter conducting polymerization and hydrolysis/condensation simultaneously, providing polymers with ceramic precursor groups followed by hydrolysis/condensation of the precursor groups, or providing a ceramic sol with polymer precursor groups and thereafter conducting one or more polymerization steps).

As indicated above, the release regions of the present invention further include an anti-restenotic agent, which may be admixed with the other components of the release region, attached one or more of the other components of the release region (e.g., the polymeric component, the optional inorganic component, etc.), or a combination thereof.

Examples of anti-restenotic agents include one or more suitable members of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) ACE inhibitors such as cilazapril, fosinopril and enalapril, (g) ATII-receptor antagonists such as saralasin and losartin, (h) platelet adhesion inhibitors such as albumin and polyethylene oxide, (i) platelet aggregation inhibitors including cilostazole, aspirin and thienopyridine (ticlopidine, clopidogrel) and GP IIb/IIIa inhibitors such as abciximab, epitifibatide and tirofiban, (j) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (k) cyclooxygenase pathway inhibitors such as aspirin, ibuprofen, flurbiprofen, indomethacin and sulfinpyrazone, (l) natural and synthetic corticosteroids such as dexamethasone, prednisolone, methprednisolone and hydrocortisone, (m) lipoxygenase pathway inhibitors such as nordihydroguairetic acid and caffeic acid, (n) leukotriene receptor antagonists, (o) antagonists of E- and P-selectins, (p) inhibitors of VCAM-1 and ICAM-1 interactions, (q) prostaglandins and analogs thereof including prostaglandins such as PGE1 and PGI2 and prostacyclin analogs such as ciprostene, epoprostenol, carbacyclin, iloprost and beraprost, (r) macrophage activation preventers including bisphosphonates, (s) HMG-CoA reductase inhibitors such as lovastatin, pravastatin, fluvastatin, simvastatin and cerivastatin, (t) fish oils and omega-3-fatty acids, (u) free-radical scavengers/antioxidants such as probucol, vitamins C and E, ebselen, trans-retinoic acid and SOD mimics, (v) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (w) MMP pathway inhibitors such as marimastat, ilomastat and metastat, (x) cell motility inhibitors such as cytochalasin B, (y) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (z) matrix deposition/organization pathway inhibitors such as halofuginone or other quinazolinone derivatives and tranilast, (aa) endothelialization facilitators such as VEGF and RGD peptide, and (bb) blood rheology modulators such as pentoxifylline.

Various preferred anti-restenotic agents may be selected from suitable members of the following, among others: paclitaxel (including particulate forms thereof, for instance, protein-bound paclitaxel particles such as albumin-bound paclitaxel nanoparticles, e.g., ABRAXANE and paclitaxel-polymer conjugates, for example, paclitaxel-poly(glutamic acid) conjugates), rapamycin (sirolimus) and its analogs (e.g., everolimus, tacrolimus, zotarolimus, etc.) as well as sirolimus-polymer conjugates and sirolimus analog-polymer conjugates such as sirolimus-poly(glutamic acid) and everolimus-poly(glutamic acid) conjugates, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2) , as well a derivatives of the forgoing, among others.

A wide range of anti-restenotic agent loadings may be used in conjunction with the medical devices of the present invention, with the therapeutically effective amount depending upon numerous factors. Typical loadings range, for example, from 1 wt % or less to 2 wt % to 5 wt % to 10 wt % to 25 wt % or more of the release region.

Numerous techniques are available for forming release regions in accordance with the present invention.

For example, where a release region is formed that contains one or more polymeric components having thermoplastic characteristics, a variety of standard thermoplastic processing techniques may be used to form the release region. Using these techniques, a release region can be formed, for instance, by (a) first providing a melt that contains polymeric component(s) as well as any other desired species (so long as they are stable under processing conditions), including optional inorganic component(s), attached or unattached anti-restenotic agent(s), attached nitric oxide producing groups (or precursors thereof, such as amine groups), etc., and (b) subsequently cooling the melt. Examples of thermoplastic processing techniques, including compression molding, injection molding, blow molding, spraying, vacuum forming and calendaring, extrusion into sheets, fibers, rods, tubes and other cross-sectional profiles of various lengths, and combinations of these processes. Using these and other thermoplastic processing techniques, entire devices or portions thereof can be made.

Other processing techniques besides thermoplastic processing techniques may also be used to form the release regions of the present invention, including solvent-based techniques. Using these techniques, a release region can be formed, for instance, by (a) first providing a solution or dispersion that contains polymeric component(s) as well as any other desired species, including optional inorganic component(s), attached or unattached anti-restenotic agent(s), attached nitric oxide producing groups or precursors thereof, etc., and (b) subsequently removing the solvent. The solvent that is ultimately selected will contain one or more solvent species, which are generally selected based on their ability to dissolve or disperse polymeric components(s) and other species that form the release region, in addition to other factors, including drying rate, surface tension, etc. Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

Numerous other techniques are also available for providing release regions that contain polymeric and inorganic sol-gel components. For example, certain techniques described above involve hydrolysis and condensation, which lead to the formation of a suspension containing a ceramic phase, which is analogous to the “sol” that is formed in sol-gel processing. In some embodiments, this suspension also includes polymeric component(s) as well as any other desired species, including attached or unattached anti-restenotic agent(s), attached nitric oxide producing groups or precursors thereof, etc. Subsequent removal of water (as well as any other solvent species that may be present), results in the formation of a solid phase, which is analogous to the “gel” in sol-gel processing. In some embodiments, such a suspension may be used to directly form a medical device or a medical device component, followed by water/solvent removal. In some embodiments, for example, where a thermoplastic polymeric component is present, the suspension may be dried and heated to form a melt for further processing. Useful techniques for processing suspensions include molding, spraying, spray coating, coating with an applicator (e.g., by roller or brush), spin-coating, dip-coating, web coating, techniques involving coating via mechanical suspension including air suspension, ink jet techniques, electrostatic techniques, molding techniques, and combinations of these processes. Useful thermoplastic techniques for processing melts are described above.

In some embodiments of the invention, a solution (where solvent-based processing is employed), a melt (where thermoplastic processing is employed) or a suspension (where sol-gel processing is employed) is applied to a substrate to form a release region. For example, the substrate can correspond to all or a portion of an implantable or insertable medical device to which a polymeric coating is applied, for example, by spraying, extrusion, and so forth. The substrate can also be, for example, a template, such as a mold, from which the release region is removed after solidification. In other embodiments, for example, extrusion and co-extrusion techniques, one or more release regions are formed without the aid of a substrate. In a specific example, an entire medical device is extruded. In another, a polymeric coating layer is co-extruded along with and underlying medical device body.

In some embodiments, for example, where the polymeric component, therapeutic agent, and optional inorganic component are charged, the release region may be formed using so-called layer-by-layer techniques in which a wide variety of substrates may be coated with charged materials via electrostatic self-assembly. In the layer-by-layer technique, a first layer having a first surface charge is typically deposited on an underlying substrate, followed by a second layer having a second surface charge that is opposite in sign to the surface charge of the first layer, and so forth. The charge on the outer layer is reversed upon deposition of each sequential layer. One specific example of a charged polymeric component, among many, is the anionically charged SIBS copolymer described in Y. A. Elabd et al., “Sulfonation and characterization of poly(styrene-isobutylene-styrene) triblock copolymers at high ion-exchange capacities,” Polymer 45 (2004) 3037-304. As a specific example of a charged polymeric component with NO releasing groups, H. Shi et al., Sensors and Actuators B 109 (2005) 341-347 describe a layer-by-layer deposition technique employing, inter alia, polyethyleneimine, which is a polymer having both primary and secondary amine groups and is sometimes represented by the formula,

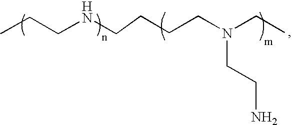

where n and m are integers. The adjacent primary amines of this compound may be reacted with NO along the following lines,

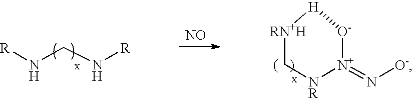

while the secondary amines become ionized at attached to a charged polymeric component include various anionic and cationic forms of paclitaxel such as paclitaxel-poly(1-glutamic acid) described in Duncan et al., Journal of Controlled Release 74 (2001)135 as well as other paclitaxel conjugates described in U.S. Pat. No. 6,730,699 to Li et al. Further information regarding layer-by-layer deposition may be found, for example, in Pub. No. US 2005/0208100 A1 to Weber et al.

In certain embodiments, the anti-restenotic agent is added to a previously formed region that comprises polymeric component(s) as well as any other desired species, including optional inorganic component(s), attached nitric oxide producing groups or precursors thereof, etc. (e.g., by imbibing).

In certain embodiments (e.g., where N-diazeniumdiolates are created), nitric oxide is added to a previously formed region that comprises polymeric component(s) as well as any other desired species, including optional inorganic component(s), anti-restenotic agent(s), attached precursors of nitric oxide producing groups, etc. (e.g., by exposure to NO at elevated pressure).

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable blood-contacting medical device comprising a release region that releases nitric oxide and an anti-restenotic agent, said release region comprising a polymeric component and an optional inorganic component, said release region comprising nitric oxide releasing groups that are attached to the polymeric component, to the optional inorganic component, or both, and said release region comprising an anti-restenotic agent, which is admixed with the polymeric and optional inorganic components, attached to the polymeric component, attached to the optional inorganic component, or a combination thereof.

2. The medical device of claim 1, wherein said anti-restenotic agent is selected from paclitaxel, polymer conjugated paclitaxel, sirolimus, polymer conjugated sirolimus, sirolimus analogs, polymer conjugated sirolimus analogs, and combinations thereof.

3. The medical device of claim 1, comprising two or more differing anti-restenotic agents.

4. The medical device of claim 1, wherein the polymeric component comprises a homopolymer.

5. The medical device of claim 1, wherein the polymeric component comprises a copolymer.

6. The medical device of claim 1, wherein the polymeric component comprises a block copolymer.

7. The medical device of claim 6, wherein the block copolymer comprises a high Tg block and a low Tg block.

8. The medical device of claim 6, wherein the block copolymer is selected from (a) a block copolymer that comprises a polyalkylene block and a polyvinyl aromatic block and (b) a polyurethane block copolymer.

9. The medical device of claim 1, wherein the release region comprises two or more differing types of polymers.

10. The medical device of claim 1, wherein the anti-restenotic agent is attached to the polymeric component.

11. The medical device of claim 1, wherein the nitric oxide releasing groups are attached to the polymeric component.

12. The medical device of claim 1, wherein the nitric oxide releasing groups comprise N-diazeniumdiolates, nitrosothiols, and combinations thereof.

13. The medical device of claim 1, wherein the release region further comprises said inorganic component.

14. The medical device of claim 13, wherein nitric oxide releasing groups are attached to the inorganic component.

15. The medical device of claim 13, wherein the anti-restenotic agent is attached to the inorganic component.

16. The medical device of claim 13, wherein the polymeric component is admixed with said inorganic component.

17. The medical device of claim 13, the polymeric component is covalently coupled to the inorganic component.

18. The medical device of claim 13, wherein the inorganic component comprises a sol-gel.

19. The medical device of claim 13, wherein the inorganic component comprises particles.

20. The medical device of claim 13, wherein the inorganic component comprises particles selected from carbon nanotubes, ceramic particles and metallic particles.

21. The medical device of claim 13, wherein the inorganic component comprises carbon nanotubes and wherein nitric oxide releasing groups are attached to the carbon nanotubes.

22. The medical device of claim 13, wherein the release region comprises two or more differing types of inorganic components.

23. The medical device of claim 1, comprising a plurality of said release regions.

24. The medical device of claim 1, wherein the release region corresponds to an entire medical device or to an entire component of a medical device.

25. The medical device of claim 1, wherein the release region is in the form of a layer that at least partially covers an underlying substrate.

26. The medical device of claim 1, wherein the medical device is a stent.

27. The medical device of claim 1, wherein the polymeric component comprises a bioerodable polymer.

28. The medical device of claim 1, wherein the nitric oxide releasing groups are attached to the polymeric component and wherein the polymeric component comprises styrene and isobutylene monomers.

29. The medical device of claim 1, wherein the release region comprises a plurality of layers of alternating charge.

30. The medical device of claim 29, wherein the release region comprises at least 10 layers of alternating charge.
